(19) United States
(12) Patent Application Publication
(10) Pub. No.: US 2026/0294414 A1
(43) Pub. Date: Oct. 1, 2026

(54) PLASTIC LOCKING MECHANISM FOR CLIP

(71) Applicant: BOSTON SCIENTIFIC SCIMED, INC., Maple Grove, MN (US)

(72) Inventor: Andrés MÁRQUEZ FERNÁNDEZ, San José de Alajuela (CR)

(21) Appl. No.: 19/465,072

(22) Filed: Jan. 30, 2026

Related U.S. Application Data
(60) Provisional application No. 63/760,443, filed on Feb. 19, 2025.

(51) Int. Cl. A61B 17/00

(52) U.S. Cl. CPC A61B 17/0057; A61B 2017/00367; A61B 2017/00584

(57) ABSTRACT

A system includes an applicator; a control member; a bushing; and a clip having a capsule coupled to the bushing and a channel, jaws, and a component coupled to the member by trapping a ball in a gap between the arms in first state. Advancing the member and the component distally to first position brings the jaws into open configuration, withdrawing them proximally from first position to second position relative to the capsule brings the jaws into closed configuration, and withdrawing them further proximally from second position to third position brings the proximal ends of the component proximal to a proximal end of the capsule permitting the arms to transition from first state to second state in which the proximal ends unflex to engage the proximal end of the capsule, lock the component in place with the jaws in closed configuration and disengage the bushing from the capsule.

### PRIORITY CLAIM

The present disclosure claims priority to U.S. Provisional Patent Application Ser. No. 63/760,443 filed Feb. 19, 2025; the disclosure of which is incorporated herewith by reference.

### BACKGROUND

Hemostasis, the process of stopping bleeding, is a critical aspect of surgical procedures and emergency medical care. Traditional methods for achieving hemostasis include manual pressure, sutures, and various types of clips and clamps. Existing hemostasis clips are designed to clamp blood vessels or tissues to prevent bleeding. While effective, hemostasis clips can be cumbersome to apply, especially in minimally invasive surgeries. Additionally, current designs may not provide adequate control over bleeding in complex anatomical regions or in cases of severe hemorrhage.

### SUMMARY

The present disclosure relates to a clipping system for treating tissue. The system includes an applicator including a flexible elongate member extending from a proximal end to a distal end which, during use, is inserted into a living body to a location adjacent to target tissue to be clipped; a control member extending through the flexible elongate member, a distal end of the control member comprising a ball; a bushing coupled to a distal end of the flexible elongate member; and a clip.

The clip includes a capsule releasably coupled to the bushing, the capsule including a channel extending therethrough; a pair of clip jaws comprising proximal ends slidably received within the channel for movement between an open configuration, in which distal ends of the clip jaws are separated from one another to receive tissue therebetween, and a closed configuration, in which the distal ends of the clip jaws are drawn toward one another to grip tissue; and a locking component slidably received within the channel, the locking component coupled to proximal portions of each clip jaw so that sliding movement of the locking component within the channel correspondingly moves the clip jaws to transition the clip jaws between the open configuration and the closed configuration.

Proximal arms of the locking component are shaped with flexible longitudinal portions and cantilevered proximal ends, the proximal arms in a first state in which the cantilevered proximal ends are in contact with an interior surface of the capsule and the longitudinal portions flex radially inward, the locking component releasably coupled to the control member by trapping the ball of the control member in a gap between the proximal arms in the first state.

Advancing the control member and the locking component distally to a first position relative to the capsule brings the clip jaws into the open configuration, withdrawing the control member and the locking component proximally from the first position to a second position relative to the capsule brings the clip jaws into the closed configuration, and withdrawing the control member and the locking component further proximally from the second position to a third position brings the cantilevered proximal ends of the locking component proximal to a proximal end of the capsule permitting the proximal arms to transition from the first state to a second state in which the proximal ends unflex to engage the proximal end of the capsule, lock the locking component in place with the clip jaws in the closed configuration and disengage the bushing from the capsule.

In an embodiment, withdrawing the control member further proximally from the third position releases the ball of the control member from the gap between the proximal arms to decouple the control member from the clip jaws.

In an embodiment, the proximal arms of the locking component include transverse portions between the longitudinal portions and the proximal ends, the transverse portions extending radially inward from the longitudinal portions and comprising an arced shape to define a rounded opening therebetween. The rounded opening changes size dependent on a degree of flexing of the longitudinal portions, a first size of the rounded opening being reduced when the proximal arms are in the first state relative to a second size of the rounded opening when the proximal arms are in the second state, the first size of the rounded opening preventing the ball from passing therethrough.

In an embodiment, withdrawing the control member and the locking component proximally from the second position to the third position causes the ball to impose a radially outward force on the proximal arms to urge the proximal arms into the second state. Withdrawing the control member further proximally from the third position causes the ball to impose a further radially outward force on the proximal arms to open the rounded opening to a third size through which the ball can pass to decouple the control member from the locking component.

In an embodiment, the locking component includes a slot extending longitudinally through a body portion of the locking component. A pin extends through the proximal portions of each of the clip jaws and the slot of the locking component to couple the clip jaws to the locking component.

In an embodiment, the slot includes an enlarged proximal portion having a diameter greater than a distal portion, the pin extending through the enlarged proximal portion when the control member and the locking component are in the first position and the second position, the distal portion of the slot having a diameter less than a diameter of the pin. Withdrawing the control member and the locking component further proximally from the second position to the third position requires a proximal force to be applied to the locking component relative to the pin to force the pin into the distal portion of the slot.

In an embodiment, the proximal end of the capsule includes a crimp comprising a crimped shape that extends radially inward relative to a remainder of the capsule, wherein the locking component in the second position is adjacent to the crimp. Withdrawing the control member and the locking component further proximally from the second position to the third position brings the proximal arms into contact with the crimp to provide a radially outward force to transition the crimp into an uncrimped shape.

In an embodiment, transitioning the crimp into the uncrimped shape disengages the bushing from the capsule and permits the proximal arms of the locking component to transition from the first state to the second state in which the proximal ends unflex to engage the proximal end of the capsule.

In an embodiment, the body portion of the locking component has a triangular shape including two angled surfaces extending from a rectangular proximal end towards one another to a distal end. Spaces between each angled surface and the interior surface of the capsule permit the clip jaws to pass into the capsule when transitioning into the closed configuration.

In addition, the present disclosure relates to a clipping system for treating tissue. The system a control member extending through a proximal portion of the system, a distal end of the control member comprising a ball; and a clip including a capsule releasably coupled to the proximal portion of the system, the capsule including a channel extending therethrough; a pair of clip jaws comprising proximal ends slidably received within the channel for movement between an open configuration, in which distal ends of the clip jaws are separated from one another to receive tissue therebetween, and a closed configuration, in which the distal ends of the clip jaws are drawn toward one another to grip tissue; and a locking component slidably received within the channel, the locking component coupled to proximal portions of each clip jaw so that sliding movement of the locking component within the channel correspondingly moves the clip jaws to transition the clip jaws between the open configuration and the closed configuration, wherein proximal arms of the locking component are shaped with flexible longitudinal portions and cantilevered proximal ends, the proximal arms in a first state in which the cantilevered proximal ends are in contact with an interior surface of the capsule and the flexible longitudinal portions flex radially inward, the locking component releasably coupled to the control member by trapping the ball of the control member in a gap between the proximal arms in the first state.

Advancing the control member and the locking component distally to a first position relative to the capsule brings the clip jaws into the open configuration, withdrawing the control member and the locking component proximally from the first position to a second position relative to the capsule brings the clip jaws into the closed configuration, and withdrawing the control member and the locking component further proximally from the second position to a third position brings the cantilevered proximal ends of the locking component proximal to a proximal end of the capsule permitting the proximal arms to transition from the first state to a second state in which the proximal ends unflex to engage the proximal end of the capsule, lock the locking component in place with the clip jaws in the closed configuration and disengage the capsule from the proximal portion of the system.

In an embodiment, withdrawing the control member further proximally from the third position releases the ball of the control member from the gap between the proximal arms to decouple the control member from the clip jaws.

In an embodiment, the proximal arms of the locking component include transverse portions between the longitudinal portions and the proximal ends, the transverse portions extending radially inward from the longitudinal portions and comprising an arced shape to define a rounded opening therebetween. The rounded opening changes size dependent on a degree of flexing of the longitudinal portions, a first size of the rounded opening being reduced when the proximal arms are in the first state relative to a second size of the rounded opening when the proximal arms are in the second state, the first size of the rounded opening preventing the ball from passing therethrough. Withdrawing the control member and the locking component proximally from the second position to the third position causes the ball to impose a radially outward force on the proximal arms to urge the proximal arms into the second state.

Withdrawing the control member further proximally from the third position causes the ball to impose a further radially outward force on the proximal arms to open the rounded opening to a third size through which the ball can pass to decouple the control member from the locking component.

In an embodiment, the locking component includes a slot extending longitudinally through a body portion of the locking component. A pin extends through the proximal portions of each of the clip jaws and the slot of the locking component to couple the clip jaws to the locking component. The slot includes an enlarged proximal portion having a diameter greater than a distal portion, the pin extending through the enlarged proximal portion when the control member and the locking component are in the first position and the second position, the distal portion of the slot having a diameter less than a diameter of the pin. Withdrawing the control member and the locking component further proximally from the second position to the third position requires a proximal force to be applied to the locking component relative to the pin to force the pin into the distal portion of the slot.

In an embodiment, the proximal end of the capsule includes a crimp comprising a crimped shape that extends radially inward relative to a remainder of the capsule, wherein the locking component in the second position is adjacent to the crimp. Withdrawing the control member and the locking component further proximally from the second position to the third position brings the proximal arms into contact with the crimp to provide a radially outward force to transition the crimp into an uncrimped shape. Transitioning the crimp into the uncrimped shape disengages the proximal portion of the system from the capsule and permits the proximal arms of the locking component to transition from the first state to the second state in which the proximal ends unflex to engage the proximal end of the capsule.

In an embodiment, the body portion of the locking component has a triangular shape including two angled surfaces extending from a rectangular proximal end towards one another to a distal end, wherein spaces between each angled surface and the interior surface of the capsule permit the clip jaws to pass into the capsule when transitioning into the closed configuration.

In addition, the present disclosure relates to a method for clipping tissue which includes inserting into a living body to a location adjacent to target tissue to be clipped a distal portion of a flexible elongate member while maintaining a proximal end of the flexible elongate member outside the living body, wherein a control member extends through an applicator and a bushing coupled to a distal end of the flexible elongate member; positioning a clip coupled to the bushing adjacent to the target tissue, the clip including a capsule releasably coupled to the bushing, the capsule including a channel extending therethrough, the clip including a pair of clip jaws comprising proximal ends slidably received within the channel for movement between an open configuration and a closed configuration, the clip including a locking component slidably received within the channel, the locking component coupled to proximal portions of each clip jaw so that sliding movement of the locking component within the channel correspondingly moves the clip jaws to transition the clip jaws between the open configuration and the closed configuration, wherein proximal arms of the locking component are shaped with flexible longitudinal portions and cantilevered proximal ends, the proximal arms in a first state in which the cantilevered proximal ends are in contact with an interior surface of the capsule and the flexible longitudinal portions flex radially inward, the locking component releasably coupled to the control member by trapping a ball of the control member in a gap between the proximal arms in the first state; advancing the control member and the locking component distally to a first position relative to the capsule bringing the clip jaws into the open configuration in which the distal ends of the clip jaws are separated from one another to receive tissue therebetween; withdrawing the control member and the locking component proximally from the first position to a second position relative to the capsule bringing the clip jaws into the closed configuration in which the distal ends of the clip jaws are drawn toward one another to grip the tissue; and withdrawing the control member and the locking component further proximally from the second position to a third position bringing the cantilevered proximal ends of the locking component proximal to a proximal end of the capsule permitting the proximal arms to transition from the first state to a second state in which the proximal ends unflex to engage the proximal end of the capsule, lock the locking component in place with the clip jaws in the closed configuration and disengage the bushing from the capsule.

In an embodiment, the method further includes withdrawing the control member further proximally from the third position releasing the ball of the control member from the gap between the proximal arms to decouple the control member from the clip jaws.

In an embodiment, the proximal arms of the locking component include transverse portions between the longitudinal portions and the proximal ends, the transverse portions extending radially inward from the longitudinal portions and comprising an arced shape to define a rounded opening therebetween. The rounded opening changes size dependent on a degree of flexing of the longitudinal portions, a first size of the rounded opening being reduced when the proximal arms are in the first state relative to a second size of the rounded opening when the proximal arms are in the second state, the first size of the rounded opening preventing the ball from passing therethrough. Withdrawing the control member and the locking component proximally from the second position to the third position causes the ball to impose a radially outward force on the proximal arms to urge the proximal arms into the second state. Withdrawing the control member further proximally from the third position causes the ball to impose a further radially outward force on the proximal arms to open the rounded opening to a third size through which the ball can pass to decouple the control member from the locking component.

In an embodiment, the locking component includes a slot extending longitudinally through a body portion of the locking component. Pin extends through the proximal portions of each of the clip jaws and the slot of the locking component to couple the clip jaws to the locking component. The slot includes an enlarged proximal portion having a diameter greater than a distal portion, the pin extending through the enlarged proximal portion when the control member and the locking component are in the first position and the second position, the distal portion of the slot having a diameter less than a diameter of the pin. Withdrawing the control member and the locking component further proximally from the second position to the third position requires a proximal force to be applied to the locking component relative to the pin to force the pin into the distal portion of the slot.

In an embodiment, the proximal end of the capsule includes a crimp comprising a crimped shape that extends radially inward relative to a remainder of the capsule, wherein the locking component in the second position is adjacent to the crimp. Withdrawing the control member and the locking component further proximally from the second position to the third position brings the proximal arms into contact with the crimp to provide a radially outward force to transition the crimp into an uncrimped shape, wherein transitioning the crimp into the uncrimped shape disengages the bushing from the capsule and permits the proximal arms of the locking component to transition from the first state to the second state in which the proximal ends unflex to engage the proximal end of the capsule.

### BRIEF DESCRIPTION OF THE DRAWINGS

FIG. 1 shows a clipping system for treating tissue defects comprising a clip releasably coupled to an applicator, the clip comprising a locking mechanism including a locking component according to various exemplary embodiments.

FIG. 2 shows the locking component of the clipping system of FIG. 1.

FIG. 3 shows the clipping system of FIG. 1 with the locking component in a first position and jaws of the clip in an open configuration.

FIG. 4 shows the clipping system of FIG. 1 with the locking component in a second position and jaws of the clip in a closed configuration.

FIG. 5 shows a rivet in an enlarged proximal portion of a slot of the locking component of FIG. 2.

FIG. 6 shows the locking component of FIG. 2 adjacent to a crimp of a capsule.

FIG. 7 shows the locking component of FIG. 2 in a third position in which arms of the locking component engage a proximal end of the capsule.

FIG. 8 shows the clipping system of FIG. 1 after deployment of the clip.

FIG. 9 shows the clipping system of FIG. 1 after deployment of the clip.

### DETAILED DESCRIPTION

The present disclosure may be further understood with reference to the following description and the appended drawings, wherein like elements are referred to with the same reference numerals. The present disclosure is directed to an endoscopic clipping system for treating internal tissue perforations, defects and/or bleeds. Exemplary embodiments of the present disclosure describe a clipping system comprising a clip releasably coupled to a proximal portion of the system, i.e., an applicator. The exemplary clip includes a pair of jaws (e.g., clip arms), proximal ends of which are slidable within a capsule to move the clip arms between an open configuration, in which distal ends of the jaws are separated from one another to receive target tissue therebetween, and a closed configuration, in which the jaws are drawn into the capsule so that the distal ends of the jaws are moved toward one another to grip the target tissue therebetween.

The clipping system includes a control member for opening and closing the jaws and for decoupling the clip from the system in a final deployment. The clipping system according to the present embodiments includes a novel mechanism for locking the clip in the closed configuration and separating the clip from the system. In particular, the exemplary locking mechanism comprises a locking component internal to the capsule shaped to include features for coupling the control member to the clip throughout initial deployment operations (including opening and closing the clip one or more times), locking the clip in the closed configuration, and decoupling the control member from the clip for final deployment.

Hemostasis, the process of stopping bleeding, is a critical aspect of surgical procedures and emergency medical care. Traditional methods for achieving hemostasis include manual pressure, sutures, and various types of clips and clamps. However, these methods often come with limitations such as difficulty in application, risk of tissue damage, and inefficiency in controlling bleeding from irregular or hard-to-reach areas. Existing hemostasis clips are designed to clamp blood vessels or tissues to prevent bleeding. Hemostasis clips generally include a distal portion (i.e., the clip) including clipping jaws, i.e., clip arms, releasably coupled to a proximal portion, i.e., an applicator, including a control member for opening and/or closing the jaws of the clip. Some existing clip designs include mechanisms for releasing the clip from the applicator which comprises a yoke and a frangible member that can be broken under tension to release the clip from the applicator.

While effective, hemostasis clips can be cumbersome to apply, especially in minimally invasive surgeries. Additionally, current designs may not provide adequate control over bleeding in complex anatomical regions or in cases of severe hemorrhage. A significant barrier to the widespread adoption of hemostasis clips is their cost. Many current designs incorporate complex locking mechanisms that are expensive to manufacture, limiting their accessibility and market reach. Furthermore, physicians often prefer hemostasis clips with a low-profile design, as this allows for better visibility and maneuverability during surgical procedures. High-profile clips can obstruct the surgical field, making it more challenging for surgeons to perform precise operations. There is a clear need for a hemostasis clip that not only provides reliable hemostatic control but also features a low-profile design and a cost-effective locking mechanism.

In various exemplary embodiments, a hemostasis clipping system comprises a locking mechanism with a compact and low-profile design. The locking mechanism generally includes a locking component internal to a capsule of the clip, the locking component coupled to proximal ends of the jaws of the clip and releasably coupled to a control member of an applicator. The locking component is shaped to retain the coupling with the control member until a final deployment in which the locking component is retracted proximally to lock the component to a proximal end of the capsule, changing the shape of the locking component to permit the control member to be decoupled from the locking component while the clip remains in place over target tissue.

The disclosed design simplifies the manufacturing process, reducing production costs while maintaining or enhancing the effectiveness and ease of use of the clip. The low-profile feature ensures better visibility and accessibility during procedures, meeting the preferences of physicians. The new mechanism is shorter than the yoke-tension breaker, allowing the capsule to be shorter as it accommodates the stroke of the jaws' opening and closing with a smaller element. By making the clip more affordable and user-friendly, the present embodiments expand the market reach of hemostasis clips, making effective hemostasis solutions available to a broader range of healthcare providers and patients. The exemplary locking component can be integrated into existing clip delivery systems, with appropriate modification to certain components. For example, the applicator of existing systems can be substantially reused with minor adjustments.

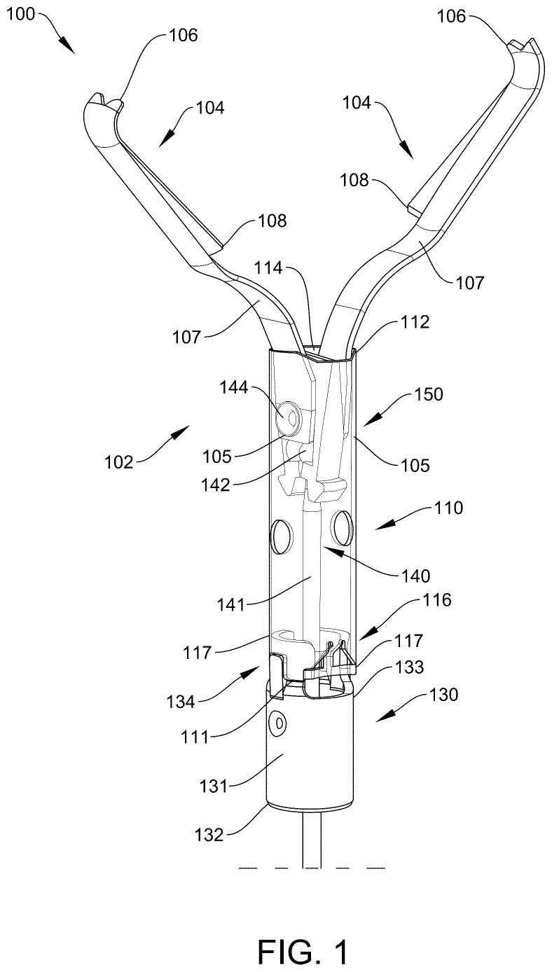

FIGS. 1-9 show a clipping system 100 for treating tissue defects comprising a clip 102 releasably coupled to a control member 140, the clip 102 comprising a locking mechanism according to various exemplary embodiments. As shown in FIG. 1, the clipping system 100 includes a clip 102 comprising a pair of jaws 104, proximal ends 105 of which are coupled to opposing sides of a locking component 150 inside a capsule 110. As shown in FIG. 1 and FIGS. 4-6, the capsule 110 is releasably coupled at its proximal end 111 to a bushing 130. As shown in FIG. 1 and FIGS. 3-7, a control member 140 extends through the bushing 130 and the capsule 110, a distal end of the control member 140 comprising a ball 142. As shown in FIG. 1 and FIGS. 3-7, the locking component 150 inside the capsule 110 is releasably coupled to the control member 140 by trapping the ball 142 in a gap 180 between proximal arms 166 of the locking component 150.

As shown in FIG. 1, the jaws 104, the capsule 110 and the locking component 150 generally comprise a distal portion of the clipping system 100, referred to collectively as the clip 102, that can be detached from a proximal portion of the clipping system 100, e.g., the bushing 130, the control member 140, and various additional components within and/or proximal to the bushing 130 such as an applicator and/or a handle (not shown). In some cases, the proximal portion of the clipping system 100 can be referred to collectively as the applicator. The control member 140 can extend through a flexible elongate member of the applicator and through a proximal portion 131 of the bushing 130. An actuator at the handle can operate the control member 140, i.e., move the control member 140 distally and proximally to open and close the jaws 104 of the clip 102, lock the locking component 150 to the capsule 110, and decouple the clip 102 from the applicator.

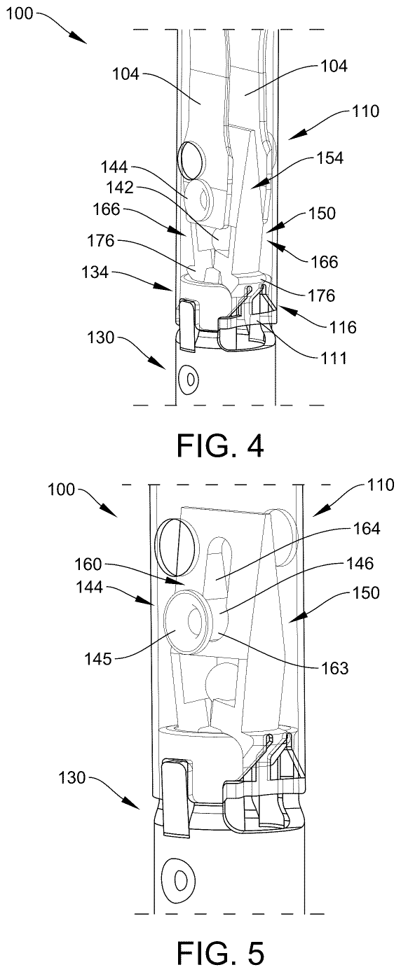

Prior to final deployment of the clip 102, the jaws 104 of the clip 102 can transition between an open configuration, as shown in FIG. 1 and FIG. 3, and a closed configuration, as shown in FIG. 4 and FIGS. 8-9. An operating physician can open or close the jaws 104 by longitudinally translating (i.e., advancing distally or withdrawing proximally) the control member 140 via the actuator at the handle. The coupling of the locking component 150 and the control member 140 via the ball 142 trapped between the arms 166 of the locking component 150 causes the locking component 150 to translate relative to the capsule 110 when the control member 140 is translated. In a first position, e.g., a distal position, the locking component 150, the ball 142 and the proximal ends 105 of the jaws 104 are located inside the capsule 110 adjacent to a distal end 112 of the capsule 110, causing the jaws 104 to be in the open configuration.

Withdrawing the control member 140 proximally from the first position draws the locking component 150 and the proximal ends 105 of the jaws 104 proximally, causing the jaws 104 to close. In a second position, e.g., a proximal position, the locking component 150, the ball 142 and the proximal ends 105 of the jaws 104 are located inside the capsule 110 adjacent to a proximal end 111 of the capsule 110, causing the jaws 104 to be in the closed configuration. In the second position, the locking component 150 remains distal to a crimp 116 on the proximal end 111 of the capsule 110. Advancing the control member 140 distally from the second position causes the jaws 104 to re-open. The operating physician can open and close the jaws 104 any number of times prior to final deployment.

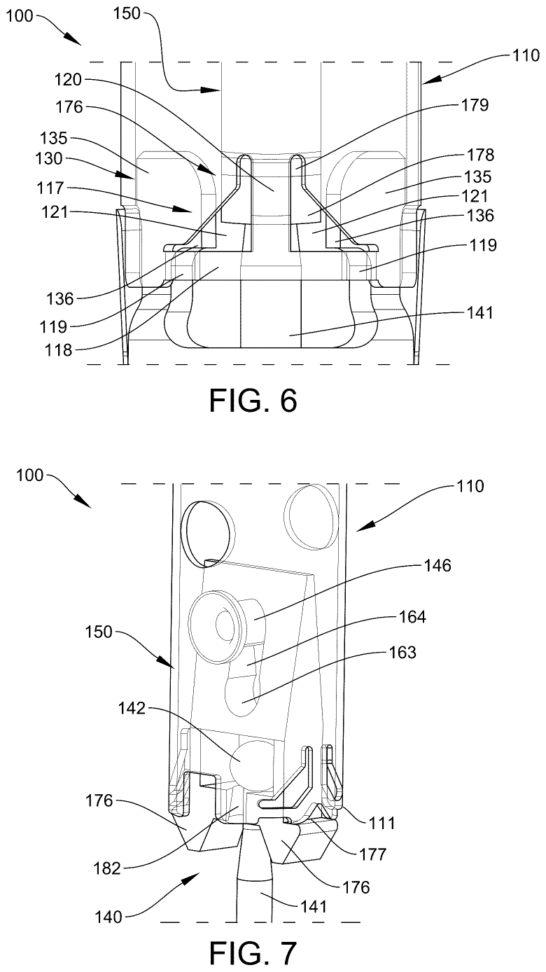

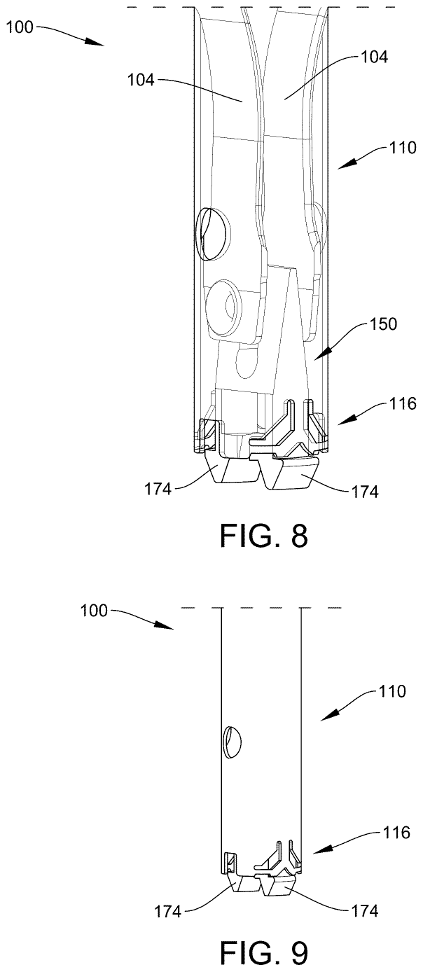

During final deployment of the clip 102, with the jaws 104 closed over target tissue, the locking component 150 and control member 140 can be withdrawn further proximally from the second position to a third position to engage the arms 166 of the locking component 150 with the crimp 116 on the proximal end 111 of the capsule 110, as shown in FIGS. 6-9. As the locking component 150 translates proximally from the second position it forces the crimp 116 open, causing the capsule 110 to detach from the bushing 130. When proximal portions 176 of the arms 166 are withdrawn to a position proximal to the crimp 116, the arms 166 will open, locking the locking component 150 in place on the proximal end 111 of the capsule 110 in the third position, as shown in FIG. 7. The control member 140 can then be withdrawn proximally through a proximal opening 182 between the arms 166 of the locking component 150, forcing the opening 182 wider by the interaction of the geometry of the ball 142 with the geometry of the arms 166. The clip 102 is fully deployed when the ball 142 of the control member 140 is withdrawn proximally separated from the locking component 150. The control member 140 can then be withdrawn from the body while the clip 102 remains in place, as shown in FIGS. 8-9.

As shown in greater detail in FIG. 1, the clip 102 includes the jaws 104, which extend from proximal ends 105 that remain within the capsule 110 to distal ends 106 that remain distal to the capsule 110. The body 107 of each jaw 104 is shaped so that, when the body 107 is distal to the capsule 110, the jaws 104 spring apart from one another (e.g., under a natural bias imparted to the jaws 104). In one embodiment, the jaws 104 are formed from heat-treated stainless steel. In another embodiment, the jaws 104 are formed of an elastic material such as nitinol and are pre-shaped (e.g., heat formed) to a shape matching a shape of the jaw 104 when in the open configuration. In any case, the jaws 104 are formed so that, when not constrained by the capsule 110 to remain in the closed configuration, the jaws 104 spring apart from one another into the open configuration.

The distal ends 106 of the jaws 104 are shaped to grip tissue positioned between the jaws 104 when the jaws 104 are transitioned from the open configuration to the closed configuration. In this example, each jaw 104 includes a feature 108 for engaging the distal end 112 of the capsule 110 to restrict further proximal movement of the clip 102 after the jaws 104 are closed. Additionally, each jaw 104 includes a proximal opening (obscured in FIG. 1) adjacent to the proximal end 105 that can be aligned with a slot 160 of the locking component 150 to couple the proximal ends 105 of the jaws 104 to the locking component 150 by a rivet 144, as described in greater detail below with regard to FIGS. 5-7.

The capsule 110 is generally cylindrical with a hollow interior (i.e., channel) comprising a proximal end 111 releasably coupled to the bushing 130 via a crimp 116 and a distal end 112 through which the jaws 104 extend. The capsule 110 can include a feature 114 at its distal end 112 to restrict proximal motion of the clip 102 after the jaws 104 are in the closed configuration. The feature 114 can be, e.g., a tab that restricts further proximal movement of the jaws 104 by interacting with the features 108 of the jaws 104. In this embodiment, the features 108 are wings that project laterally from the jaws 104 to a width greater than a width of an opening at the tab. Thus, the features 108 abut against the tab when the clip is drawn into the capsule 110 to the maximum extent.

The proximal end 111 of the capsule 110 comprises a crimp 116 including two side portions 117 shaped so that the crimp 116 can transition from a crimped state, in which each side portion 117 comprises a shape that extends in a direction at least partially radially inward (relative to the cross-section of the remainder of the capsule 110), to an uncrimped state, in which each side portion 117 of the crimp 116 is forced radially outward into a shape that opens the proximal end 111 of the capsule 110, e.g., so that the capsule 110 at the proximal end 111 has a cross-section similar to the remainder of the capsule 110, to be described in greater detail below with regard to FIGS. 6-9. Transitioning the crimp 116 to the uncrimped state permits the clip 102 to detach from the bushing 130 in a final deployment of the clip 102, to be described in greater detail below.

The bushing 130 functions to ensure that the clip 102 remains coupled to the applicator (not shown) during insertion and initial deployment operations and completely separates from the applicator after the user has deployed the clip 102. Specifically, it is important that the applicator be completely separated from the clip 102 after deployment to prevent a situation in which the clip 102 is locked and gripped over tissue in the body while the applicator remains connected to the clip 102. As would be understood by those skilled in the art, such failure to separate the applicator from the clip 102 may require a separate procedure to separate the clip 102 from either the tissue over which it is clipped or from the applicator.

In this embodiment, the bushing 130 includes a proximal portion 131 comprising a proximal end 132 fixedly coupled to a flexible elongate member and a distal end 133 coupled to a distal portion 134 of the bushing 130. In some embodiments, the distal portion 134 can comprise a moving coupler that is slidingly coupled to the proximal portion 131 including a pair of distally extending wings 135 with notches 136 for engaging locking features of the crimp 116 of the capsule 110, to be described in greater detail below with regard to FIG. 6.

As would be understood by those skilled in the art, the bushing 130 and the capsule 110 of this embodiment are both generally cylindrical. However, the shape of these components can be varied in any manner desired so long as the applicator and the clip 102 may be inserted through a working channel of a delivery device (e.g., flexible endoscope) and manipulated as desired without damaging the delivery device.

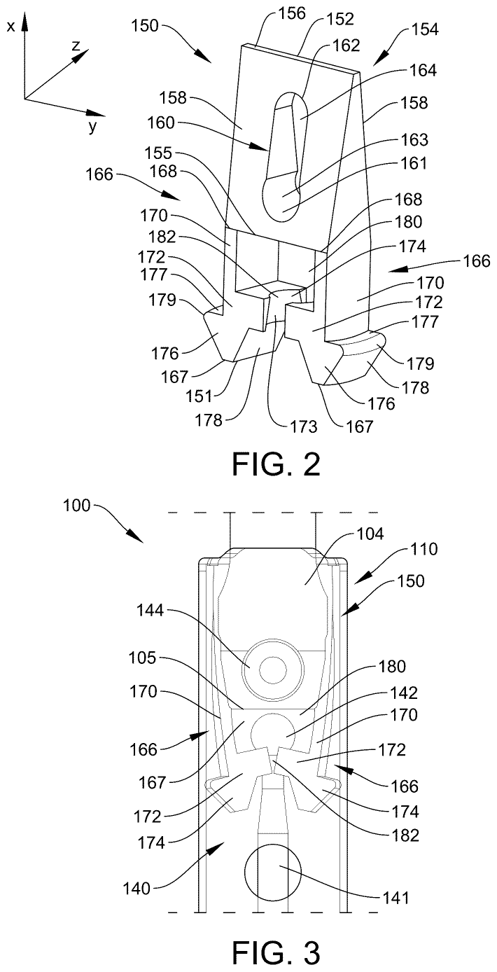

As shown in greater detail in FIG. 2, the locking component 150 is formed into a particular shape that can be manufactured, e.g., by injection molding, 3D printing, etc., as a unitary component. The locking component 150 generally comprises a body portion 154 distal to two arms 166. A proximal end 151 of the locking component 150 corresponds to proximal ends 167 of the arms 166 and a distal end 152 of the locking component 150 corresponds to a distal end 156 of the body portion 154. For ease of description, FIG. 2 includes an axis indicator showing an x-axis, y-axis and z-axis, wherein the x-axis corresponds to the longitudinal axis of the clip 102, and the y-axis and z-axis correspond to axes transverse to the longitudinal axis.

Dimensions of the locking component 150 in the direction of the x-axis are referred to as lengths; dimensions of the locking component 150 in the direction of the y-axis are referred to as widths; and dimensions of the locking component 150 in the direction of the z-axis are referred to as depths.

The body portion 154 is generally shaped similar to a triangular prism. A proximal end 155 of the body portion 154 generally comprises a rectangular proximal surface (not shown) (e.g., in the yz-plane) and connections to distal ends 168 of the arms 166. The width of the proximal end 155 of the body portion 154 (y-direction) is greater than the depth of the proximal end 155 (z-direction). The body portion 154 comprises a three-dimensional volume defined in part by the rectangular proximal end 155 and by two surfaces 158 extending from the proximal end 155 longitudinally and at slight opposing angles (relative to the x-axis) toward one another so that the surfaces 158 meet (or nearly meet) at a rounded or thin rectangular surface at the distal end 156 of the body portion 154. In other words, the depth of the body portion 154 is at a maximum at the proximal end 155 and decreases to a minimum at the distal end 156. The generally triangular shape of the body portion 154 provides space between the surfaces 158 and the interior surface of the capsule 110 so that, when the locking component 150 is translated proximally to transition the clip 102 from the open configuration (e.g., as shown in FIG. 1) to the closed configuration (e.g., as shown in FIG. 4), the body 107 of the jaws 104 is better able to conform to the interior volume of the capsule 110.

In this embodiment, a slot 160 extends longitudinally through the body portion 154 from a proximal end 161 to a distal end 162. The depth of the slot 160 varies according to the cross-section of the body portion 154, i.e., the depth of the slot 160 is at its maximum at the proximal end 161 and decreases to a minimum at the distal end 162. The slot 160 comprises an enlarged proximal portion 163 adjacent to the proximal end 161, the enlarged proximal portion 163 having a greater diameter (e.g., in the xy-plane) than a distal portion 164 having a uniform diameter extending distally from the enlarged proximal portion 163 to the distal end 162.

As previously discussed, the locking component 150 and the jaws 104 of the clip 102 are coupled by a rivet 144 extending (in order) through a proximal opening of a first jaw of the jaws 104, the slot 160, and a proximal opening of a second jaw of the jaws 104. In particular, during insertion and initial deployment operations, the rivet 144 extends through the enlarged proximal portion 163 of the slot 160. The enlarged proximal portion 163 is shaped to snugly receive the pin 146 of the rivet 144, as shown in greater detail in FIG. 5. In other words, the pin 146 of the rivet 144 and the enlarged proximal portion 163 of the slot 160 have similar diameters, while the distal portion 164 of the slot 160 has a reduced diameter. The pin 146 can pass into the distal portion 164 when a sufficient proximal force is applied to the locking component 150 relative to the pin 146, such that resistive forces are overcome (e.g., the pin 146 is deformed radially inward (i.e., squeezed) and/or the uniform distal portion 164 is deformed to a greater diameter (i.e., opened)), as shown in greater detail in FIG. 7. The slot 160 is shaped as such so that the rivet 144 remains within the enlarged proximal portion 163 (i.e., in a proximal position relative to the locking component 150) until the operating physician decides to fully deploy the clip 102, at which time a further proximal force is imparted on the locking component 150 by the control member 140 to bring the rivet 144 into the uniform distal portion 164 of the slot 160, to be described in greater detail below with regard to FIGS. 5-7.

The arms 166 of the locking component 150 extend proximally off the proximal end 155 of the body portion 154. The arms 166 are formed into a shape that can be generally described as including a longitudinal portion 170, a transverse portion 172, and a proximal portion 176. The distal ends 168 of the arms 166 correspond to distal ends of the longitudinal portion 170, the proximal ends 167 of the arms 166 correspond to proximal ends of the proximal portion 176, and the transverse portion 172 is between the longitudinal portion 170 and the proximal portion 176. The proximal ends 167 of the arms 166 are cantilevered.

The longitudinal portion 170 generally comprises a shape similar to a rectangular prism, having a depth (z-direction) corresponding to the depth of the proximal end 155 of the body portion and a width (y-direction) substantially smaller than the depth, permitting the cantilevered arms 166 to flex. The distal ends 168 of the arms 166 are located on opposing sides of the rectangular proximal end 155 of the body portion 154 and are oriented so that proximal ends 167 of the arms 166 can flex toward one another or away from one another (e.g., in the xy-plane).

The transverse portion 172 generally comprises a shape similar to a rectangular prism with recesses 174 formed by an inner surface 173 thereof. In particular, the transverse portions 172 extend toward one another (e.g., along the y-axis) and the recesses 174 are formed in the inner surfaces 173 facing one another. The inner surfaces 173 are arced, e.g., in a semicircular shape, such that a rounded opening 182 (e.g., a circular opening) is formed between the arms 166, the rounded opening 182 including the recesses 174 and space therebetween.

A gap 180 is defined within the locking component 150 by the proximal surface of the proximal end 155 of the body portion 154, the inner surfaces of the longitudinal portions 170 of the arms 166, and the distal surfaces of the transverse portions 172. The gap 180 is sized and shaped to receive the ball 142 of the control member 140 therewithin. When the arms 166 are flexed inward (toward one another), the opening 182 between the arms 166 is reduced such that the ball 142 is trapped in the gap 180, as shown in FIG. 3. In this configuration, the control member 140 is coupled to the locking component 150 so that a user can, by an actuator at the handle (not shown), move the locking component 150 longitudinally within the capsule 110 to open and close the jaws 104. When the arms 166 are flexed outward (away from one another), the opening 182 between the arms 166 is increased such that the ball 142 is permitted to exit the gap 180 through the opening 182, as shown in FIG. 7. The opening 182 is slightly smaller than the ball 142, so the user has to apply some force to remove the ball 142. This enables the mechanism to force the arms 166 to open even more.

The proximal portion 176 of each arm 166 generally comprises a shape similar to a triangular prism. A distal end of the proximal portion 176 (in the yz-plane) can be considered rectangular and the proximal portion 176 comprises a three-dimensional volume defined in part by the rectangular distal end and by two surfaces 178 extending from the distal end longitudinally and at opposing angles (relative to the x-axis) toward one another so that the surfaces 178 meet (or nearly meet) at a rounded or thin rectangular surface at the proximal ends 167 of the proximal portion 176. The proximal portion 176 is formed so that that a first portion of its distal end is connected to the transverse portion 172 and a second portion of its distal end is outside (e.g., along the y-axis) the remainder of the locking component 150, e.g., extends radially outward relative to the longitudinal axis. The exposed part of the distal end (e.g., a distal surface 177) of the proximal portion 176 connects to the surface 178 by a rounded edge 179.

The shape of the locking component 150 as shown in FIG. 2 corresponds to a manufactured shape of the locking component 150 under no external forces, e.g., an unbiased shape. As described above, the arms 166 of the locking component 150 can flex inward when a radially inward force is imparted upon the arms 166 (e.g., upon the transverse portion 172 or the proximal portion 176 of the arms 166) or can flex outward when a radially outward force is imparted upon the arms 166.

The locking component 150 is sized so that the width of the locking component 150 (in the y-direction) (in particular, the width of the body portion 154 and/or the distance between opposing outer surfaces of the longitudinal portion 170 and the transverse portion 172 of the arms 166) is only slightly less than an inner diameter of the capsule 110, while the distance between the rounded edges 179 of the proximal portions 176 is greater than the inner diameter of the capsule 110. Accordingly, to assemble the clipping system 100 with the locking component 150 inside the capsule 110, the arms 166 are required to deform (flex) inwards.

As shown in greater detail in FIG. 3, the clipping system 100 is assembled so that the ball 142 of the control member 140 is received within the gap 180 interior to the locking component 150 when the locking component 150 is brought within the capsule 110. The rounded edges 179 of the proximal portions 176 of the arms 166 are brought into contact with the interior surface of the capsule 110, such that the arms 166 are flexed inward, i.e., in a deformed state. In the deformed state, the opening 182 between the arms 166 is decreased such that the ball 142 is trapped in the gap 180. The control member 140 remains coupled to the clip 102 so long as the proximal portions 176 of the arms 166 remain within the capsule 110. The jaws 104 are coupled to the locking component 150 by the rivet 144 extending through the proximal openings in the jaws 104 and the enlarged proximal portion 163 of the slot 160.

In FIG. 1 and FIG. 3, the locking component 150, control member 140 (e.g., the ball 142 at the distal end thereof), and proximal ends 105 of the jaws 104 are in a first position within the capsule 110 (a distal-most position) such that the jaws 104 are in the open configuration. Withdrawing the control member 140 proximally can bring the locking component 150, control member 140 (e.g., the ball 142 at the distal end thereof), and proximal ends 105 of the jaws 104 to a second position within the capsule 110 (a proximal position) such that the jaws 104 are in the closed configuration. The jaws 104 are not permitted to move further proximally from the second position.

As shown in greater detail in FIG. 4, in the second position, the jaws 104 are closed and the proximal portions 176 of the arms 166 are adjacent to the crimp 116 on the proximal end 111 of the capsule 110. As shown in greater detail in FIG. 5, in the second position, the rivet 144 remains in the enlarged proximal portion 163 of the slot 160. When the clip 102 is in its closed configuration, the locking component 150 is impeded from further proximal motion by the resistance generated by the crimp 116 and by the diameter difference of the slot 160. If no greater proximal force is applied to overcome these forces, the clip 102 is allowed to return to the open configuration. To deploy and lock the clip 102 in place, a greater proximal closing force must be applied to overcome the resistance of the crimp 116 and the diameter difference in the slot 160.

As shown in greater detail in FIG. 6, the system 100 is assembled with each side portion 117 of the crimp 116 in a crimped state. In the crimped state, the crimp 116 includes features for locking the capsule 110 to the bushing 130. Each side portion 117 of the crimp 116 generally comprises a transverse portion 118 and a longitudinal portion 120 with openings 121 defined between these portions and the remainder of the capsule 110 permitting the transition between the crimped state and the uncrimped state, as would be understood by those skilled in the art. In the crimped state, the longitudinal portion 120 is forced radially inward to form an angled surface, as shown in the views of FIGS. 4-5. The transverse portion 118 is forced radially inward, forming radially inward parts 119 for engaging the notches 136 of the bushing 130. Accordingly, with the crimp in the crimped state, the capsule 110 is coupled to the bushing 130.

The crimp 116 is shaped so that the crimp 116 can transition between a crimped state and an uncrimped state. In particular, a radially outward force imparted on the crimp 116 causes the crimp 116 to straighten and disengage from the bushing 130, as shown in FIG. 7. The radially outward force can be imparted on the crimp 116 by the locking component 150, in particular, the proximal portion 176 of the arms 166. As shown in FIG. 6, when the locking component 150 is brought to the second position, the proximal portions 176 of the arms 166 are brought into contact with the crimp 116, in particular, the longitudinal portion 120 thereof. The geometry of the proximal portions 176 of the arms guide the locking component 150 to align with the crimp 116 using the borders of the bushing 130. The rounded edge 179 of each of the proximal portions 176 is adjacent to a distal end of a corresponding part of the longitudinal portion 120 of the crimp 116. Drawing the locking component 150 further proximally causes the rounded edge 179 to force the corresponding longitudinal portion 120 of the crimp 116 radially outward to straighten the crimp 116 into the uncrimped state.

As described above, when the locking component 150 is in this position shown in FIG. 6, the jaws 104 are not permitted to move further proximally. However, the slot 160 of the locking component 150 permits the locking component 150 to move further proximally while the jaws 104 remain in position in the closed configuration. To draw the locking component 150 further proximally requires a force sufficient to overcome the crimp 116 and to force the rivet 144 proximally past the change in diameter of the slot 160. As the locking component 150 moves proximally, it forces the crimp 116 to open radially outward, detaching the bushing 130 from the capsule 110. Due to the geometry of the locking component 150, when the ball 142 is forced against the inner surface 173 of the transverse portion 172 of the locking component 150, it also forces the arms 166 to open, making it easier to open the crimp 116.

As shown in greater detail in FIG. 7, when the proximal portions 176 of the arms 166 of the locking component 150 are brought proximally beyond the proximal end 111 of the capsule 110, the arms 166 are no longer constrained by the interior geometry of the capsule 110 permitting the arms 166 to unflex into a state similar to the manufactured state of the locking component 150 in which the arms 166 (e.g., the longitudinal portions 170 thereof) extend substantially longitudinally and the proximal portions 176, particularly, the distal surfaces 177 thereof, are moved radially outward beyond the diameter of the capsule 110. This third position of the locking component 150 is a locked position. No further proximal motion is permitted by the locking component 150 relative to the capsule 110 due to the geometry of the jaws 104 when they are closed (e.g., the features 108 of the jaws 104 engaging the feature 114 at the distal end 112 of the capsule 110), and distal motion is prevented by the interference of the distal surfaces 177 of the proximal portion 176 against the proximal end 111 of the capsule 110.

From the third position, the control member 140 can be retracted further proximally to decouple the control member 140 from the locking component 150 (and from the clip 102). The ball 142 on the distal end of the control member 140 in this embodiment has a diameter slightly larger than the diameter of the opening 182 between the transverse portions 172 of the arms 166. The geometry of the ball 142 interacting with the inner surfaces 173 of the transverse portion 172 can force the arms 166 to flex radially outwardly (along with the crimp 116), permitting the ball 142 to exit the gap 180. In particular, the distal part of the opening 182 has a diameter slightly smaller than the ball 142. The opening 182 has a tapered form with an angle such that the proximal part of the opening 182 has a bigger diameter. This is done so that when the arms 166 of the locking component 150 are bent inwards the opening 182 is able to accommodate freely the control member 140. The control member 140 and the bushing 130 can then be retracted proximally from the body, leaving clip 102 deployed in the body, as shown in FIGS. 8-9.

Several resistance forces are involved in the functioning of opening, closing, and deploying the clip, and balancing these forces is crucial for its correct operation. The forces involved in the operations include a closing force (Fc), an opening force (Fo), a lower arm frictional force (Ff), a slot resistance force (Fs), a crimp opening force (Fcr), and a ball release force (Fd). The closing force, Fc, comprises a resistance force exerted by the jaws 104 while they are coming inside the capsule 110. The opening force, Fo, comprises a force required to bring the jaws 104 out of the capsule 110. The lower arms frictions force, Ff, comprises a friction force between the rounded edge 179 of the proximal portions 176 of the locking component 150 and the capsule 110. The slot resistance force, Fs, comprises a force that needs to be overcome to move the pin 146 of the rivet 144 through the slot 160. The crimp opening force, Fcr, comprises the force required to open the crimp 116 in the capsule 110. The ball release force, Fd, comprises the force required for the ball 142 of the control member 140 to detach from the locking component 150. In some embodiments, Fc, Fo, and Ff should be similar so enhance an ergonomic operation of the handle. In some embodiments, Fs and Fcr should be greater than Fc, Fo, or Ff, e.g., Fs +Fcr >Fc +Fo +Ff. In general, Fd should be ergonomically acceptable so that the user can operate the device accurately and without undue fatigue.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather modifications are also covered within the scope of the present invention as defined by the appended claims. Specifically, although this application describes various embodiments each having different features in various combinations, those skilled in the art will understand that any of the features of one embodiment may be combined with the features of the other embodiments in any manner not specifically disclaimed or which is not functionally or logically inconsistent with the operation of the device or the stated functions of the disclosed embodiments.

1-15. (canceled)

16. A clipping system for treating tissue, comprising:
an applicator including a flexible elongate member extending from a proximal end to a distal end which, during use, is inserted into a living body to a location adjacent to target tissue to be clipped;
a control member extending through the flexible elongate member, a distal end of the control member comprising a ball;
a bushing coupled to a distal end of the flexible elongate member; and
a clip including:
a capsule releasably coupled to the bushing, the capsule including a channel extending therethrough;
a pair of clip jaws comprising proximal ends slidably received within the channel for movement between an open configuration, in which distal ends of the clip jaws are separated from one another to receive tissue therebetween, and a closed configuration, in which the distal ends of the clip jaws are drawn toward one another to grip tissue; and
a locking component slidably received within the channel, the locking component coupled to proximal portions of each clip jaw so that sliding movement of the locking component within the channel correspondingly moves the clip jaws to transition the clip jaws between the open configuration and the closed configuration, wherein proximal arms of the locking component are shaped with flexible longitudinal portions and cantilevered proximal ends, the proximal arms in a first state in which the cantilevered proximal ends are in contact with an interior surface of the capsule and the longitudinal portions flex radially inward, the locking component releasably coupled to the control member by trapping the ball of the control member in a gap between the proximal arms in the first state,
wherein advancing the control member and the locking component distally to a first position relative to the capsule brings the clip jaws into the open configuration, withdrawing the control member and the locking component proximally from the first position to a second position relative to the capsule brings the clip jaws into the closed configuration, and withdrawing the control member and the locking component further proximally from the second position to a third position brings the cantilevered proximal ends of the locking component proximal to a proximal end of the capsule permitting the proximal arms to transition from the first state to a second state in which the proximal ends unflex to engage the proximal end of the capsule, lock the locking component in place with the clip jaws in the closed configuration and disengage the bushing from the capsule.

17. The clipping system of claim 16, wherein withdrawing the control member further proximally from the third position releases the ball of the control member from the gap between the proximal arms to decouple the control member from the clip jaws.

18. The clipping system of claim 17, wherein the proximal arms of the locking component include transverse portions between the longitudinal portions and the proximal ends, the transverse portions extending radially inward from the longitudinal portions and comprising an arced shape to define a rounded opening therebetween,
wherein the rounded opening changes size dependent on a degree of flexing of the longitudinal portions, a first size of the rounded opening being reduced when the proximal arms are in the first state relative to a second size of the rounded opening when the proximal arms are in the second state, the first size of the rounded opening preventing the ball from passing therethrough.

19. The clipping system of claim 18, wherein withdrawing the control member and the locking component proximally from the second position to the third position causes the ball to impose a radially outward force on the proximal arms to urge the proximal arms into the second state,
wherein withdrawing the control member further proximally from the third position causes the ball to impose a further radially outward force on the proximal arms to open the rounded opening to a third size through which the ball can pass to decouple the control member from the locking component.

20. The clipping system of claim 16, wherein the locking component includes a slot extending longitudinally through a body portion of the locking component,
wherein a pin extends through the proximal portions of each of the clip jaws and the slot of the locking component to couple the clip jaws to the locking component.

21. The clipping system of claim 20, wherein the slot includes an enlarged proximal portion having a diameter greater than a distal portion, the pin extending through the enlarged proximal portion when the control member and the locking component are in the first position and the second position, the distal portion of the slot having a diameter less than a diameter of the pin,
wherein withdrawing the control member and the locking component further proximally from the second position to the third position requires a proximal force to be applied to the locking component relative to the pin to force the pin into the distal portion of the slot.

22. The clipping system of claim 21, wherein the proximal end of the capsule includes a crimp comprising a crimped shape that extends radially inward relative to a remainder of the capsule, wherein the locking component in the second position is adjacent to the crimp,
wherein withdrawing the control member and the locking component further proximally from the second position to the third position brings the proximal arms into contact with the crimp to provide a radially outward force to transition the crimp into an uncrimped shape.

23. The clipping system of claim 22, wherein transitioning the crimp into the uncrimped shape disengages the bushing from the capsule and permits the proximal arms of the locking component to transition from the first state to the second state in which the proximal ends unflex to engage the proximal end of the capsule.

24. The clipping system of claim 20, wherein the body portion of the locking component has a triangular shape including two angled surfaces extending from a rectangular proximal end towards one another to a distal end,
wherein spaces between each angled surface and the interior surface of the capsule permit the clip jaws to pass into the capsule when transitioning into the closed configuration.

25. A clipping system for treating tissue, comprising:
a control member extending through a proximal portion of the system, a distal end of the control member comprising a ball; and
a clip including:
a capsule releasably coupled to the proximal portion of the system, the capsule including a channel extending therethrough;
a pair of clip jaws comprising proximal ends slidably received within the channel for movement between an open configuration, in which distal ends of the clip jaws are separated from one another to receive tissue therebetween, and a closed configuration, in which the distal ends of the clip jaws are drawn toward one another to grip tissue; and
a locking component slidably received within the channel, the locking component coupled to proximal portions of each clip jaw so that sliding movement of the locking component within the channel correspondingly moves the clip jaws to transition the clip jaws between the open configuration and the closed configuration, wherein proximal arms of the locking component are shaped with flexible longitudinal portions and cantilevered proximal ends, the proximal arms in a first state in which the cantilevered proximal ends are in contact with an interior surface of the capsule and the flexible longitudinal portions flex radially inward, the locking component releasably coupled to the control member by trapping the ball of the control member in a gap between the proximal arms in the first state,
wherein advancing the control member and the locking component distally to a first position relative to the capsule brings the clip jaws into the open configuration, withdrawing the control member and the locking component proximally from the first position to a second position relative to the capsule brings the clip jaws into the closed configuration, and withdrawing the control member and the locking component further proximally from the second position to a third position brings the cantilevered proximal ends of the locking component proximal to a proximal end of the capsule permitting the proximal arms to transition from the first state to a second state in which the proximal ends unflex to engage the proximal end of the capsule, lock the locking component in place with the clip jaws in the closed configuration and disengage the capsule from the proximal portion of the system.

26. The clipping system of claim 25, wherein withdrawing the control member further proximally from the third position releases the ball of the control member from the gap between the proximal arms to decouple the control member from the clip jaws.

27. The clipping system of claim 26, wherein the proximal arms of the locking component include transverse portions between the longitudinal portions and the proximal ends, the transverse portions extending radially inward from the longitudinal portions and comprising an arced shape to define a rounded opening therebetween,
wherein the rounded opening changes size dependent on a degree of flexing of the longitudinal portions, a first size of the rounded opening being reduced when the proximal arms are in the first state relative to a second size of the rounded opening when the proximal arms are in the second state, the first size of the rounded opening preventing the ball from passing therethrough,
wherein withdrawing the control member and the locking component proximally from the second position to the third position causes the ball to impose a radially outward force on the proximal arms to urge the proximal arms into the second state,
wherein withdrawing the control member further proximally from the third position causes the ball to impose a further radially outward force on the proximal arms to open the rounded opening to a third size through which the ball can pass to decouple the control member from the locking component.

28. The clipping system of claim 27, wherein the locking component includes a slot extending longitudinally through a body portion of the locking component,
wherein a pin extends through the proximal portions of each of the clip jaws and the slot of the locking component to couple the clip jaws to the locking component,
wherein the slot includes an enlarged proximal portion having a diameter greater than a distal portion, the pin extending through the enlarged proximal portion when the control member and the locking component are in the first position and the second position, the distal portion of the slot having a diameter less than a diameter of the pin,
wherein withdrawing the control member and the locking component further proximally from the second position to the third position requires a proximal force to be applied to the locking component relative to the pin to force the pin into the distal portion of the slot.

29. The clipping system of claim 28, wherein the proximal end of the capsule includes a crimp comprising a crimped shape that extends radially inward relative to a remainder of the capsule, wherein the locking component in the second position is adjacent to the crimp,
wherein withdrawing the control member and the locking component further proximally from the second position to the third position brings the proximal arms into contact with the crimp to provide a radially outward force to transition the crimp into an uncrimped shape,
wherein transitioning the crimp into the uncrimped shape disengages the proximal portion of the system from the capsule and permits the proximal arms of the locking component to transition from the first state to the second state in which the proximal ends unflex to engage the proximal end of the capsule.

30. The clipping system of claim 28, wherein the body portion of the locking component has a triangular shape including two angled surfaces extending from a rectangular proximal end towards one another to a distal end, wherein spaces between each angled surface and the interior surface of the capsule permit the clip jaws to pass into the capsule when transitioning into the closed configuration.

31. A method for clipping tissue, comprising:
inserting into a living body to a location adjacent to target tissue to be clipped a distal portion of a flexible elongate member while maintaining a proximal end of the flexible elongate member outside the living body, wherein a control member extends through an applicator and a bushing coupled to a distal end of the flexible elongate member;
positioning a clip coupled to the bushing adjacent to the target tissue, the clip including a capsule releasably coupled to the bushing, the capsule including a channel extending therethrough, the clip including a pair of clip jaws comprising proximal ends slidably received within the channel for movement between an open configuration and a closed configuration, the clip including a locking component slidably received within the channel, the locking component coupled to proximal portions of each clip jaw so that sliding movement of the locking component within the channel correspondingly moves the clip jaws to transition the clip jaws between the open configuration and the closed configuration, wherein proximal arms of the locking component are shaped with flexible longitudinal portions and cantilevered proximal ends, the proximal arms in a first state in which the cantilevered proximal ends are in contact with an interior surface of the capsule and the flexible longitudinal portions flex radially inward, the locking component releasably coupled to the control member by trapping a ball of the control member in a gap between the proximal arms in the first state;
advancing the control member and the locking component distally to a first position relative to the capsule bringing the clip jaws into the open configuration in which the distal ends of the clip jaws are separated from one another to receive tissue therebetween;
withdrawing the control member and the locking component proximally from the first position to a second position relative to the capsule bringing the clip jaws into the closed configuration in which the distal ends of the clip jaws are drawn toward one another to grip the tissue; and
withdrawing the control member and the locking component further proximally from the second position to a third position bringing the cantilevered proximal ends of the locking component proximal to a proximal end of the capsule permitting the proximal arms to transition from the first state to a second state in which the proximal ends unflex to engage the proximal end of the capsule, lock the locking component in place with the clip jaws in the closed configuration and disengage the bushing from the capsule.

32. The method of claim 31, further comprising:
withdrawing the control member further proximally from the third position releasing the ball of the control member from the gap between the proximal arms to decouple the control member from the clip jaws.

33. The method of claim 32, wherein the proximal arms of the locking component include transverse portions between the longitudinal portions and the proximal ends, the transverse portions extending radially inward from the longitudinal portions and comprising an arced shape to define a rounded opening therebetween,
wherein the rounded opening changes size dependent on a degree of flexing of the longitudinal portions, a first size of the rounded opening being reduced when the proximal arms are in the first state relative to a second size of the rounded opening when the proximal arms are in the second state, the first size of the rounded opening preventing the ball from passing therethrough,
wherein withdrawing the control member and the locking component proximally from the second position to the third position causes the ball to impose a radially outward force on the proximal arms to urge the proximal arms into the second state,
wherein withdrawing the control member further proximally from the third position causes the ball to impose a further radially outward force on the proximal arms to open the rounded opening to a third size through which the ball can pass to decouple the control member from the locking component.

34. The method of claim 31, wherein the locking component includes a slot extending longitudinally through a body portion of the locking component,
wherein a pin extends through the proximal portions of each of the clip jaws and the slot of the locking component to couple the clip jaws to the locking component,
wherein the slot includes an enlarged proximal portion having a diameter greater than a distal portion, the pin extending through the enlarged proximal portion when the control member and the locking component are in the first position and the second position, the distal portion of the slot having a diameter less than a diameter of the pin,
wherein withdrawing the control member and the locking component further proximally from the second position to the third position requires a proximal force to be applied to the locking component relative to the pin to force the pin into the distal portion of the slot.

35. The method of claim 34, wherein the proximal end of the capsule includes a crimp comprising a crimped shape that extends radially inward relative to a remainder of the capsule, wherein the locking component in the second position is adjacent to the crimp,
wherein withdrawing the control member and the locking component further proximally from the second position to the third position brings the proximal arms into contact with the crimp to provide a radially outward force to transition the crimp into an uncrimped shape, wherein transitioning the crimp into the uncrimped shape disengages the bushing from the capsule and permits the proximal arms of the locking component to transition from the first state to the second state in which the proximal ends unflex to engage the proximal end of the capsule.
